(19) United States
(12) Patent Application Publication
(10) Pub. No.: US 2004/0253283 A1
(43) Pub. Date: Dec. 16, 2004

(54) Composition for treating keratinous materials, comprising a hydroxycarboxylic acid and a protective and/or conditioning agent

(75) Inventors: Rainer Muller, Leopoldshafen (DE); Patricia Desenne, Bois Colombes (FR)

Correspondence Address: LERNER, DAVID, LITTENBERG,, KRUMHOLZ & MENTLIK, 600 SOUTH AVENUE WEST, WESTFIELD, NJ, 07090, US

(73) Assignee: L'OREAL S.A., Paris (FR)

(21) Appl. No.: 10/809,851

(22) Filed: Mar. 25, 2004

Related U.S. Application Data
(60) Provisional application No. 60/461,211, filed on Apr. 8, 2003.

(30) Foreign Application Priority Data
Mar. 25, 2003 (FR) 0303643

(51) Int. Cl. A61K 31/19; A61K 7/00

(52) U.S. Cl. 424/401; 514/557

(57) ABSTRACT

The invention relates to a composition for treating a keratinous material, preferably the hair, comprising, a cosmetically acceptable medium, at least one of protective and conditioning agent, and at least one hydroxycarboxylic acid, or a salt thereof. These combinations make it possible to improve the deposition of the agent for protecting or conditioning a keratinous material, and also the cosmetic properties.

### CROSS-REFERENCE TO RELATED APPLICATIONS

The present application claims the benefit of French Application No. 0303643, filed Mar. 25, 2003, and U.S. Provisional Application No. 60/461,211, filed Apr. 8, 2003, the disclosures of which are incorporated by reference herein.

The invention relates to a composition for treating keratinous materials, preferably human keratinous fibers such as the hair, comprising, in a physiologically and preferably cosmetically acceptable medium, at least one of protective and conditioning agent, and at least one particular hydroxycarboxylic acid, or salts thereof.

### BACKGROUND OF THE INVENTION

It is well known that hair is sensitized or embrittled to varying degrees by the action of atmospheric agents and especially of light, water and humidity, and also by the repeated action of various hair treatments such as washing, permanent-waving, relaxing, dyeing and bleaching. Many publications disclose that natural light destroys certain amino acids of the hair. Since these attacks impair the hair fiber, they reduce its mechanical properties, for instance the tensile strength, the breaking load and the elasticity, or its resistance to swelling in an aqueous medium. The hair then becomes dull, coarse and brittle. In contrast with the skin, the hair becomes lighter in colour.

It is also known that the light and washing agents have a tendency to attack the natural colour of the hair, and also the artificial colour of dyed hair. The colour of the hair gradually fades or changes to shades that are unattractive or undesirable.

Substances for protecting the hair against the degradations caused by atmospheric attack, such as the light and heat, and treatments, have been sought for many years in the cosmetics industry. Preferably, products are sought that protect the colour of naturally coloured or artificially dyed keratinous fibers and that preserve or reinforce the intrinsic mechanical properties of the keratinous fibers (tensile strength, breaking load and elasticity, or their resistance to swelling in an aqueous medium).

To combat these degradations of hair keratin, it has already been proposed to use certain substances capable of screening out light radiation, for instance 2-hydroxy-4-methoxybenzophenone-5-sulphonic acid or salts thereof (FR-A-2 627 085), 4-(2-oxo-3-bornylidenemethyl)benzenesulphonic acid or salts thereof (EP-A-329 032) or lactoferrin (FR-A-2 673 839).

However, when these screening agents are efficient, they are only efficient at high concentrations. However, at these concentrations, hair treated with these screening agents has a coarse, laden feel. Furthermore, such hair is extremely difficult to disentangle.

It has already been recommended, in compositions for washing or caring for keratinous materials such as the hair, to use conditioning agents, preferably cationic polymers or silicones, to facilitate the disentangling of the hair and to give it softness and suppleness. However, the cosmetic advantages mentioned above are unfortunately also accompanied, on dried hair, by certain cosmetic effects considered undesirable, namely heaviness of the hairstyle (lack of lightness of the hair), and lack of smoothness (hair that is not uniform from the root to the tip).

In addition, the use of cationic polymers for this purpose presents various drawbacks. On account of their high affinity for the hair, some of these polymers become deposited in large amounts during repeated use, and lead to adverse effects such as an unpleasant, laden feel, stiffening of the hair or adhesion between the fibers, which affects the styling. These drawbacks are accentuated in the case of fine hair, which lacks liveliness and volume.

It is well known that hair that has been sensitized (i.e., damaged and/or embrittled) to varying degrees by the action of atmospheric agents or by the action of mechanical or chemical treatments, such as dyeing, bleaching and/or permanent-waving, is often difficult to disentangle and to style, and lacks softness.

In summary, it is found that the current cosmetic compositions containing protective and/or conditioning agents are not entirely satisfactory.

Moreover, cosmetic compositions generally contain a complexing agent for complexing the metal cations liable to be present in trace amounts in these compositions, and also those that may be present on the hair and that originate from the ambient air, from the water with which this hair has been washed, or from shampoos or other hair products with which the hair has been treated. Specifically, it is very important to neutralize these metal cations, since they are capable of catalysing the oxidation reactions of hair fibers, and of doing so in an uncontrolled manner, which may be reflected by severe adverse effects such as brittleness of the hair or burning of the scalp.

### DETAILED DESCRIPTION

The complexing agents most commonly used at the present time in oxidizing compositions for dyeing, bleaching or permanently reshaping keratinous fibers are ethylenediaminetetraacetic acid (EDTA) and its derivatives, for instance diethylenetriaminepentaacetic acid (DPTA), generally in weight proportions of about from 0.1% to 1%.

However, in the context of its research, the Applicant has found that EDTA and derivatives thereof have insufficient complexing properties in compositions of this type. These findings, which are corroborated by the results obtained by other research teams, justify the search for novel complexing agents.

### SUMMARY OF THE INVENTION

A complexing agent intended to form part of the constitution of cosmetic compositions must satisfy numerous requirements. Specifically, in addition to the fact that it must have high complexing power with respect to metals, so as to eliminate or, at the very least, minimize the risk of catalysis of oxidation reactions on the keratinous fibers by the metallic traces liable to be present in these compositions and on these fibers, it must be compatible, and preferably must not react, with the other constituents. It must also be stable in solution. It must also be totally harmless to these fibers and to the skin, and must preferably be lacking in any allergenic nature.

The Applicant has now discovered that the combination of a particular complexing agent with protective and/or conditioning agents makes it possible to increase the deposition of the agent for protecting or conditioning keratinous materials and thereby to increase the protection or conditioning.

An improvement in the cosmetic properties is observed, such as, preferably, the ease of styling, the hold, the liveliness or the volume of the treated hair.

All these discoveries form the basis of the present invention.

Thus, according to the present invention, novel cosmetic compositions are now proposed, comprising, in a physiologically and preferably cosmetically acceptable medium, at least one particular agent for protecting or conditioning keratinous materials and at least one particular hydroxycarboxylic acid or salts thereof.

One preferred embodiment of the invention is thus a composition for treating keratinous materials, preferably human keratinous fibers such as the hair, comprising, in a physiologically and preferably cosmetically acceptable medium, at least one hydroxycarboxylic acid or salts thereof corresponding to the general formula (I) below:

R—(CHOH)₄—CO₂X (I)

in which:

R represents a group CO₂X, and

X represents a hydrogen atom or a monovalent or divalent cation derived from a transition metal, from an alkali metal or alkaline-earth metal, from an organic amine or from an ammonium ion,

and at least one of protective agent and conditioning agent, the said conditioning agent being chosen from synthetic oils, plant oils, fluoro or perfluoro oils, natural or synthetic waxes, silicones, non-polysaccharide cationic polymers, compounds of ceramide type, cationic surfactants, fatty amines, fatty acids and derivatives thereof, and also mixtures of these various compounds.

Another preferred embodiment of the invention relates to the use of at least one particular hydroxycarboxylic acid or salts thereof in, or for the manufacture of, a cosmetic composition comprising an agent for protecting or conditioning keratinous materials.

Yet another preferred embodiment of the invention is also the use of at least one particular hydroxycarboxylic acid or salts thereof in a composition comprising an agent for protecting or conditioning keratinous materials, to increase the efficacy of this agent for protecting or conditioning keratinous materials.

Another preferred embodiment of the present invention is also the use of at least one particular hydroxycarboxylic acid or salts thereof in a composition comprising an agent for protecting or conditioning keratinous materials, to improve the deposition and/or fixing of the said protective and/or conditioning agent on the keratinous materials.

### DETAILED DESCRIPTION OF THE INVENTION

The various preferred embodiments of the invention will now be detailed. All the meanings and definitions of the compounds used in the present invention, which are given below, are valid for all of the preferred embodiments of the invention.

The particular hydroxycarboxylic acids or salts thereof correspond to the general formula (I) below:

R—(CHOH)₄—CO₂X (I)

in which:

R represents a group CO₂X, and

X represents a hydrogen atom or a monovalent or divalent cation derived from a transition metal, from an alkali metal or alkaline-earth metal, from an organic amine or from an ammonium ion.

Thus, the complexing agents used in the context of the invention correspond to hydroxycarboxylic acids and to the corresponding carboxylates.

Since formula (I) comprises 4 groups of chiral H—C—OH atoms, it is known to those skilled in the art that this formula includes enantiomers, diastereoisomers, racemic mixtures, and other mixtures of which would satisfy this formula.

In accordance with the invention, the monovalent or divalent cation is preferably chosen from the group consisting of alkali metal cations, alkaline-earth metal cations and divalent transition metal cations.

Examples of alkali metal cations that may preferably be mentioned include sodium (Na⁺) and potassium (K⁺), while examples of alkaline-earth metal cations that may preferably be mentioned include calcium (Ca²⁺) and magnesium (Mg²⁺).

For the purposes of the present invention, the term “transition metal” means a metal comprising an incomplete d subshell, preferably in oxidation state II, such as cobalt (Co²⁺), iron (Fe²⁺), manganese (Mn²⁺), zinc (Zn²⁺) and copper (Cu²⁺)

The compound(s) of formula (I) is (are) preferably chosen from the group consisting of mucic acid (C₆H₁₀O₈)—also known as galactaric acid—glucaric acid (C₆H₁₀O₈), mannaric acid (C₆H₁₀O₈), the alkali metal salts thereof, the alkaline-earth metal salts thereof, the transition metal salts thereof, and mixtures thereof, for instance mixtures of mucic acid and of sodium mucate (C₆H₈O₈Na₂).

In a preferred embodiment, the compound(s) of formula (I) is (are) mucic acid.

Preferably, the compound(s) of formula (I) represent(s) from 0.001% to 10% by weight, and more preferably from 0.001% to 5% by weight relative to the total weight of the composition.

The agents for protecting keratinous materials may be any active agent that is useful for preventing or limiting degradation caused by physical or chemical attack.

Thus, the agent for protecting keratinous materials may be chosen from water-soluble, liposoluble or water-insoluble organic UV-screening agents, free-radical scavengers, antioxidants, vitamins or provitamins.

The organic UV-screening agents (systems for screening out UV radiation) are chosen preferably from silicone or non-silicone, water-soluble or liposoluble or water insoluble screening agents, or mineral oxide nanoparticles whose surface has optionally been treated to make them hydrophilic or hydrophobic.

The water-soluble organic UV-screening agents may be chosen, for example, from para-aminobenzoic acid or its salts, anthranilic acid or its salts, salicylic acid or its salts, p-hydroxycinnamic acid or its salts, sulphonic derivatives of benz-x-azoles (benzothiazoles, benzimidazoles and benzoxazoles) or salts thereof, sulphonic derivatives of benzopheflone or salts thereof, sulphonic derivatives of benzylidenecamphor or salts thereof, benzylidenecamphor derivatives substituted with a quaternary amine or salts thereof, phthalylidene-camphorsulphonic acid derivatives or salts thereof, sulphonic derivatives of benzotriazole, or mixtures thereof.

It is also possible to use hydrophilic polymers having, in addition and on account of their chemical nature, UV-photoprotective properties. Mention may be made of polymers comprising benzylidenecamphor and/or benzotriazole groups, substituted with sulphonic or quaternary ammonium groups.

As liposoluble (or lipophilic) organic UV-screening agents that are suitable for use in the present invention, mention may be made preferably of: p-aminobenzoic acid derivatives, such as p-aminobenzoic acid esters or amides; salicylic acid derivatives such as the esters; benzophenone derivatives; dibenzoylmethane derivatives; diphenylacrylate derivatives; benzofuran derivatives; polymeric UV-screening agents containing one or more organosilicon residues; cinnamic acid esters; camphor derivatives; trianilino-s-triazine derivatives; urocanic acid ethyl ester; benzotriazoles; hydroxyphenyltriazine derivatives; bis-resorcinol-dialkylaminotriazines; and mixtures thereof.

The liposoluble (or lipophilic) UV-screening agent according to the invention is preferably chosen from: octyl salicylate; 4-tert-butyl-4′-methoxydibenzoylmethane (Parsol 1789 from Givaudan); octocrylene; 2-ethylhexyl 4-methoxycinnamate (Parsol MCX) and the compound of formula (II) below, or 2-(2H-benzotriazol-2-yl)-4-methyl-6-[2-methyl-3-[1,3,3,3-tetramethyl-1-[(trimethylsilyl)oxy]disiloxanyl]-propynyl]phenol, described in patent application EP-A-0 392 883:

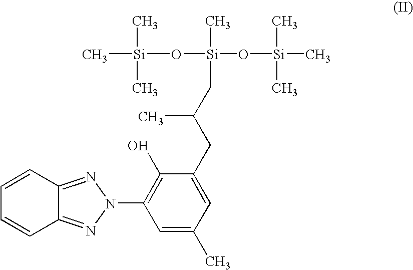

Other UV-screening agents that are particularly preferred according to the invention are benzophenone derivatives such as Uvinul MS 40 (2-hydroxy-4-methoxybenzophenone-5-sulphonic acid) and Uvinul M40 (2-hydroxy-4-methoxybenzophenone) sold by BASF, benzalmalonate derivatives such as Parsol SLX (polydimethyl/methyl (3-(4-(2,2-bis-ethoxycarbonylvinyl)phenoxy)propenyl)siloxane) sold by Givaudan-Rouré, benzylidenecamphor derivatives such as Mexoryl SX (β,β′-camphorsulphonic [1,4-divinylbenzene] acid) manufactured by the company Chimex, and benzimidazole derivatives such as Eusolex 232 (2-phenylbenzimidazole-5-sulphonic acid) sold by Merck.

The mineral oxides may be chosen from titanium oxides, zinc oxides or cerium oxides.

The antioxidants and/or free-radical scavengers are chosen preferably from phenols such as BHA (tert-butyl-4-hydroxyanisole), BHT (2,6-di-tert-butyl-p-cresol), TBHQ (tert-butylhydroquinone), polyphenols such as proanthocyanidol oligomers and flavonoids, hindered amines known under the generic term HALS (Hindered Amine Light Stabilizer) such as tetraminopiperidine, erythorbic acid, polyamines such as spermine, cysteine, glutathione, superoxide dismutase and lactoferrin.

The vitamins are chosen preferably from ascorbic acid, vitamin E, vitamin E acetate, B vitamins such as vitamins B3 and B5, vitamin PP, or vitamin A or its derivatives.

The provitamins are chosen preferably from panthenol and retinol.

According to the invention, the agent(s) for protecting keratinous materials may represent from 0.001% to 20% by weight, preferably from 0.01% to 10% by weight and more preferably from 0.1% to 5% by weight, relative to the total weight of the final composition.

In the context of the present patent application, the term “conditioning agent” means any agent whose function is to improve the cosmetic properties of the hair, preferably the softness, disentangling, feel, smoothness or static electricity.

The conditioning agents may be in liquid, semi-solid or solid form such as, for example, oils, waxes or gums.

According to the invention, the conditioning agents may be chosen from synthetic oils such as polyolefins, plant oils, fluoro oils or perfluoro oils, natural or synthetic waxes, silicones, non-polysaccharide cationic polymers, compounds of ceramide type, cationic surfactants, fatty amines, fatty acids or derivatives thereof, or mixtures of these various compounds.

The conditioning agents that are preferred according to the invention are cationic polymers or silicones.

The synthetic oils are preferably polyolefins, preferably poly-α-olefins and more preferably:

of hydrogenated or non-hydrogenated polybutene type, and preferably hydrogenated or non-hydrogenated polyisobutene type.

Isobutylene oligomers with a molecular weight of less than 1000 and mixtures thereof with polyisobutylenes with a molecular weight of greater than 1000, and more preferably between 1000 and 15 000, are preferably used.

As examples of poly-α-olefins that can be used in the context of the present invention, mention may be made preferably of the polyisobutenes sold under the name Permethyl 99 A, 101 A, 102 A, 104 A (n=16) and 106 A (n=38) by the company Presperse Inc., or alternatively the products sold under the name Arlamol HD (n=3) by the company ICI (n denoting the degree of polymerization),

of hydrogen ated or non-hydrogenated polydecene type.

Such products are sold, for example, under the names Ethylflo by the company Ethyl Corp. and Arlamol PAO by the company ICI.

The animal or plant oils are preferably chosen from the group formed by sunflower oil, corn oil, soybean oil, avocado oil, jojoba oil, marrow oil, grapeseed oil, sesame oil, hazelnut oil, fish oils, glyceryl tricaprocaprylate, or plant or animal oils of formula R₉COOR₁₀ in which R₉ represents a higher fatty acid residue containing from 7 to 29 carbon atoms and R₁₀ represents a linear or branched hydrocarbon-based chain containing from 3 to 30 carbon atoms, preferably alkyl or alkenyl, for example purcellin oil or liquid jojoba wax.

It is also possible to use natural or synthetic essential oils such as, for example, eucalyptus oil, lavendin oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil.

The waxes are natural (animal or plant) or synthetic substances that are solid at room temperature (20°-25° C.). They are insoluble in water, soluble in oils and are capable of forming a water-repellent film.

For the definition of waxes, mention may be made, for example, of P. D. Dorgan, Drug and Cosmetic Industry, December 1983, pp. 30-33.

The wax(es) is (are) chosen preferably from carnauba wax, candelilla wax, alfalfa wax, paraffin wax, ozokerite, plant waxes such as olive tree wax, rice wax, hydrogenated jojoba wax or the absolute waxes of flowers such as the essential wax of blackcurrant flower sold by the company Bertin (France), animal waxes such as beeswaxes, or modified beeswaxes (cerabellina); other waxes or waxy starting materials which can be used according to the invention are, for example, marine waxes such as the product sold by the company Sophim under the reference M82, and polyethylene waxes or polyolefin waxes in general.

The non-saccharide cationic polymers that may be used in accordance with the present invention may be chosen from all those already known per se as improving the cosmetic properties of hair treated with detergent compositions, i.e., preferably those described in patent application EP-A-0 337 354 and in French patent applications FR-A-2 270 846, 2 383 660, 2 598 611, 2 470 596 and 2 519 863.

The term “non-saccharide polymers” is understood to mean polymers that do not contain a glycoside bond between monosaccharides.

Even more generally, for the purpose of the present invention, the term “cationic polymer” denotes any polymer containing cationic groups and/or groups that may be ionized into cationic groups.

The cationic polymers that are preferred are chosen from those containing units comprising primary, secondary, tertiary and/or quaternary amine groups that either may form part of the main polymer chain or may be borne by a side substituent directly attached thereto.

The cationic polymers used generally have a number-average molecular mass of between 500 and 5×10⁶ approximately and preferably between 103 and 3×10⁶ approximately.

Among the cationic polymers that may be mentioned preferably are polymers of the polyamine, polyamino amide and polyquaternary ammonium type. These are known products.

The polymers of the polyamine, polyamino amide and polyquaternary ammonium type that may be used in accordance with the present invention, and that may preferably be mentioned, are those described in French Patents 2 505 348 and 2 542 997. Among these polymers, mention may be made of:

(1) homopolymers or copolymers derived from acrylic or methacrylic esters or amides and comprising at least one of the units of the following formulae:

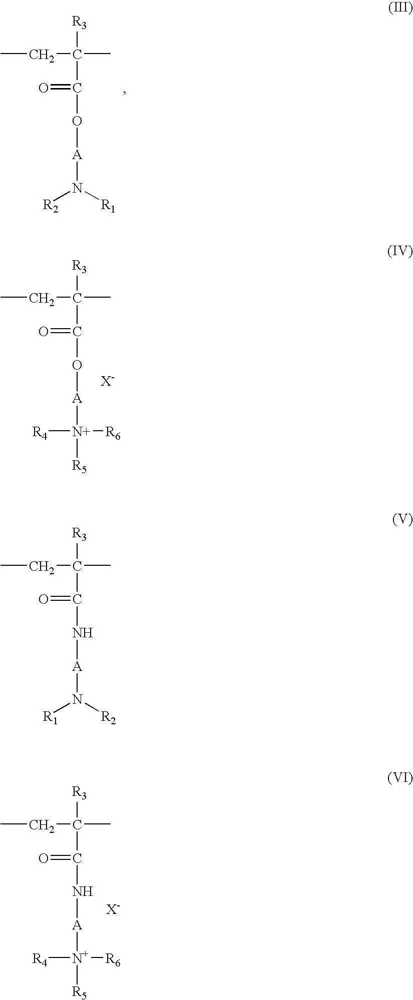

in which:

R₁ and R₂, which may be identical or different, represent hydrogen or an alkyl group containing from 1 to 6 carbon atoms, and preferably methyl or ethyl;

R₃, which may be identical or different, denote a hydrogen atom or a CH₃ radical;

A, which may be identical or different, represent a linear or branched alkyl group of 1 to 6 carbon atoms, preferably 2 or 3 carbon atoms, or a hydroxyalkyl group of 1 to 4 carbon atoms;

R₄, R₅ and R₆, which may be identical or different, represent an alkyl group containing from 1 to 18 carbon atoms or a benzyl radical and preferably an alkyl group containing from 1 to 6 carbon atoms;

X denotes an anion derived from a mineral or organic acid, such as a methosulphate anion or a halide such as chloride or bromide.

The copolymers of family (1) can also contain one or more units derived from comonomers which may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these copolymers of family (1), mention may be made of:

copolymers of acrylamide and of dimethylaminoethyl methacrylate quaternized with dimethyl sulphate or with a dimethyl halide, such as the product sold under the name Hercofloc by the company Hercules,

the copolymers of acrylamide and of methacryloyloxyethyltrimethylammonium chloride described, for example, in patent application EP-A-080 976 and sold under the name Bina Quat P 100 by the company Ciba Geigy,

the copolymer of acrylamide and of methacryloyloxyethyltrimethylammonium methosulphate sold under the name Reten by the company Hercules,

quaternized or non-quaternized vinylpyrrolidone/dialkylaminoalkyl acrylate or methacrylate copolymers, such as the products sold under the name Gafquat by the company ISP, such as, for example, Gafquat 734 or Gafquat 755, or alternatively the products known as Copolymer 845, 958 and 937. These polymers are described in detail in French Patents 2 077 143 and 2 393 573,

dimethylaminoethyl methacrylate/vinylcaprolactam/vinylpyrrolidone terpolymers, such as the product sold under the name Gaffix VC 713 by the company ISP,

vinylpyrrolidone/methacrylamidopropyldimethylamine copolymers sold preferably under the name Styleze CC 10 by ISP, and

quaternized vinylpyrrolidone/dimethylaminopropyl-methacrylamide copolymers, such as the product sold under the name Gafquat HS 100 by the company ISP.

(2) Polymers consisting of piperazinyl units and of divalent alkylene or hydroxyalkylene radicals containing straight or branched chains, optionally interrupted by oxygen, sulphur or nitrogen atoms or by aromatic or heterocyclic rings, as well as the oxidation and/or quaternization products of these polymers. Such polymers are described, preferably, in French Patents 2 162 025 and 2 280 361.

(3) Water-soluble polyamino amides prepared preferably by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they contain one or more tertiary amine functions, they can be quaternized. Such polymers are described, preferably, in French Patents 2 252 840 and 2 368 508.

(4) The polyamino amide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical contains from 1 to 4 carbon atoms and preferably denotes methyl, ethyl or propyl. Such polymers are described preferably in French patent 1 583 363.

Among these derivatives, mention may be made more preferably of the adipic acid/dimethylamino-hydroxypropyl/diethylenetriamine polymers sold under the name Cartaretine F, F4 or F8 by the company Sandoz.

(5) The polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid is between 0.8:1 and 1.4:1; the polyamino amide resulting therefrom being reacted with epichlorohydrin in a molar ratio of epichlorohydrin relative to the secondary amine group of the polyamino amide of between 0.5:1 and 1.8:1. Such polymers are described preferably in U.S. Pat. Nos. 3,227,615 and 2,961,347.

Polymers of this type are sold preferably under the name Hercosett 57 by the company Hercules Inc. or alternatively under the name PD 170 or Delsette 101 by the company Hercules in the case of the adipic acid/epoxypropyl/diethylenetriamine copolymer.

(6) Cyclopolymers of alkyldiallylamine or of dialkyldiallylammonium, such as the homopolymers or copolymers containing, as main constituent of the chain, units corresponding to formula (VII) or (VIII):

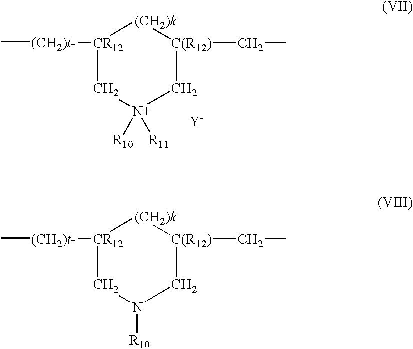

in which formulae k and t are equal to 0 or 1, the sum k+t being equal to 1; R₁₂ denotes a hydrogen atom or a methyl radical; R₁₀ and R₁₁, independently of each other, denote an alkyl group having from 1 to 6 carbon atoms, a hydroxyalkyl group in which the alkyl group preferably has 1 to 5 carbon atoms, a lower (C₁-C₄) amidoalkyl group, or R₁₀ and R₁₁ can denote, together with the nitrogen atom to which they are attached, heterocyclic groups such as piperidyl or morpholinyl; Y⁻ is an anion such as bromide, chloride, acetate, borate, citrate, tartrate, bisulphate, bisulphite, sulphate or phosphate. These polymers are described preferably in French patent 2 080 759 and in its Certificate of Addition 2 190 406.

R₁₀ and R₁₁, independently of each other, preferably denote an alkyl group containing from 1 to 4 carbon atoms.

Among the polymers defined above, mention may be made preferably of the dimethyldiallylammonium chloride homopolymer sold under the name Merquat 100 by the company Nalco (and its homologues of low weight-average molecular mass) and the copolymers of diallyldimethylammonium chloride and of acrylamide, sold under the name Merquat 550.

(7) The quaternary diammonium polymer containing repeating units corresponding to the formula:

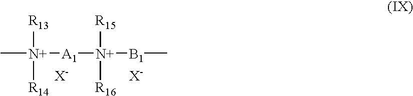

in which formula (IX):

R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally containing a second hetero atom other than nitrogen, or alternatively R₁₃, R₁₄, R₁₅ and R₁₆ represent a linear or branched C₁-C₆ alkyl radical substituted with a nitrile, ester, acyl or amide group or a group —CO—O—R₁₇-D or —CO—NH—R₁₇-D where R₁₇ is an alkylene and D is a quaternary ammonium group;

A₁ and B₁ represent polymethylene groups containing from 2 to 20 carbon atoms which may be linear or branched, saturated or unsaturated, and which may contain, linked to or intercalated in the main chain, one or more aromatic rings or one or more oxygen or sulphur atoms or sulphoxide, sulphone, disulphide, amino, alkylamino, hydroxyl, quaternary ammonium, ureido, amide or ester groups, and

X⁻ denotes an anion derived from a mineral or organic acid;

A₁, R₁₃ and R₁₅ can form, with the two nitrogen atoms to which they are attached, a piperazine ring; in addition, if A₁ denotes a linear or branched, saturated or unsaturated alkylene or hydroxyalkylene radical, B₁ can also denote a group (CH₂)ₙ—CO-D-OC—(CH₂)ₙ—, n being an integer ranging from about 2 to 20,

in which D denotes:

a) a glycol residue of formula: —O-Z-O—, where Z denotes a linear or branched hydrocarbon-based radical or a group corresponding to one of the following formulae:

—(CH₂—CH₂—O)ₓ—CH₂—CH₂—

—[CH₂—CH(CH₃)—O]_{y}—CH₂—CH(CH₃)—

where x and y denote an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, where Y denotes a linear or branched hydrocarbon-based radical, or alternatively the divalent radical —CH₂—CH₂—S—S—CH₂—CH₂—;

d) a ureylene group of formula: —NH—CO—NH—.

Preferably, X⁻ is an anion such as chloride or bromide.

These polymers generally have a number-average molecular mass of between 1000 and 100 000.

Polymers of this type are described preferably in French patents 2 320 330, 2 270 846, 2 316 271, 2 336 434 and 2 413 907 and U.S. Pat. Nos. 2,273,780; 2,375,853; 2,388,614; 2,454,547; 3,206,462; 2,261,002; 2,271,378; 3,874,870; 4,001,432; 3,929,990; 3,966,904; 4,005,193; 4,025,617; 4,025,627; 4,025,653; 4,026,945; and 4,027,020.

It is more preferred to use polymers that consist of repeating units corresponding to the formula:

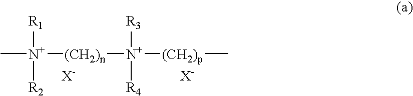

in which R₁, R₂, R₃ and R₄, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

A compound of formula (a) that is particularly preferred is the compound for which R₁, R₂, R₃ and R₄ represent a methyl radical and n=3, p=6 and X=Cl, referred to as hexadimethrine chloride according to the INCI nomenclature (CTFA).

(8) Polyquaternary ammonium polymers consisting of units of formula (X):

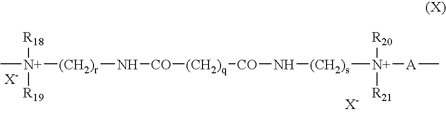

in which formula:

R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical,

where p is equal to 0 or to an integer between 1 and 6, with the proviso that R₁₈, R₁₉, R₂₀ and R₂₁ do not simultaneously represent a hydrogen atom,

r and s, which may be identical or different, are integers ranging from 1 to 6,

q is equal to 0 or to an integer ranging from 1 to 34,

X⁻ denotes an anion such as a halide,

A denotes a divalent radical or preferably represents —CH₂—CH₂—O—CH₂—CH₂—.

Such compounds are described in patent application EP-A-122 324.

Among these products, mention may be made, for example, of Mirapol® A 15, Mirapol® AD1, Mirapol® AZ1 and Mirapol® 175 sold by the company Miranol.

(9) Quaternary polymers of vinylpyrrolidone and of vinylimidazole, such as, for example, the products sold under the names Luviquat® FC 905, FC 550 and FC 370 by the company BASF.

(10) Crosslinked methacryloyloxy(C₁-C₄)alkyltri-(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, preferably methylenebisacrylamide. A crosslinked acrylamide/methacryloyloxyethyltrimethylammonium chloride copolymer (20/80 by weight) in the form of a dispersion containing 50% by weight of the said copolymer in mineral oil can be used more preferably. This dispersion is sold under the name Salcare® SC 92 by the company Ciba. A crosslinked methacryloyloxyethyltrimethylammonium chloride homopolymer containing about 50% by weight of the homopolymer in mineral oil or in a liquid ester can also be used. These dispersions are sold under the names Salcare® SC 95 and Salcare® SC 96 by the company Ciba.

Other cationic polymers that can be used in the context of the invention are cationic proteins or cationic protein hydrolysates, polyalkyleneimines, preferably polyethyleneimines, polymers containing vinylpyridine or vinylpyridinium units, condensates of polyamines and of epichlorohydrin, quaternary polyureylenes and chitin derivatives.

Among all the cationic polymers that may be used in the context of the present invention, it is preferred to use cationic cyclopolymers, preferably the dimethyldiallylammonium chloride homopolymers or copolymers sold under the names Merquat 100, Merquat 550 and Merquat S by the company Nalco, and quaternary vinylpyrrolidone and vinylimidazole polymers, and mixtures thereof.

The silicones that may be used in accordance with the invention are preferably polyorganosiloxanes that are insoluble in the composition and that may be in the form of oils, waxes, resins or gums.

The organopolysiloxanes are defined in greater detail in Walter Noll's “Chemistry and Technology of Silicones” (1968) Academic Press. They can be volatile or non-volatile.

When they are volatile, the silicones are more preferably chosen from those having a boiling point of between 60° C. and 260° C., and even more preferably from:

(i) cyclic silicones containing from 3 to 7 and preferably 4 to 5 silicon atoms. These are, for example, octamethylcyclotetrasiloxane sold preferably under the name Volatile Silicone 7207 by Union Carbide or Silbione 70045 V 2 by Rhodia Chimie, decamethylcyclopentasiloxane sold under the name Volatile Silicone 7158 by Union Carbide, and Silbione 70045 V 5 by Rhodia Chimie, and mixtures thereof.

Mention may also be made of cyclocopolymers of the dimethylsiloxanes/methylalkylsiloxane type, such as Volatile Silicone FZ 3109 sold by the company Union Carbide, having the chemical structure:

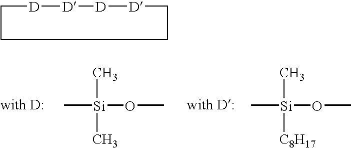

Mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones containing 2 to 9 silicon atoms and having a viscosity of less than or equal to 5×10⁻⁶ m²/s at 25° C. An example is decamethyltetrasiloxane sold preferably under the name SH 200 by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Non-volatile silicones, and more preferably polyalkylsiloxanes, polyarylsiloxanes, polyalkylarylsiloxanes, silicone gums and resins, polyorganosiloxanes modified with organofunctional groups, and mixtures thereof, are preferably used.

These silicones are more preferably chosen from polyalkylsiloxanes, among which mention may be made mainly of polydimethylsiloxanes containing trimethylsilyl end groups having a viscosity of from 5×10⁻⁶ to 2.5 m²/s at 25° C. and preferably 1×10⁻⁵ to 1 m²/s. The viscosity of the silicones is measured, for example, at 25° C. according to ASTM standard 445 Appendix C.

Among these polyalkylsiloxanes, mention may be made, in a non-limiting manner, of the following commercial products:

the Silbione oils of the 47 and 70 047 series or the Mirasil oils sold by Rhodia Chimie, such as, for example, the oil 70 047 V 500 000;

the oils of the Mirasil series sold by the company Rhodia Chimie;

the oils of the 200 series from the company Dow Corning, such as, more preferably, DC200 with a viscosity of 60 000 cSt;

the Viscasil oils from General Electric and certain oils of the SF series (SF 96, SF 18) from General Electric.

Mention may also be made of polydimethylsiloxanes containing dimethylsilanol end groups (Dimethiconol according to the CTFA name) such as the oils of the 48 series from the company Rhodia Chimie.

In this category of polyalkylsiloxanes, mention may also be made of the products sold under the names Abil Wax 9800 and 9801 by the company Goldschmidt, which are poly (C₁-C₂₀) alkylsiloxanes.

The polyalkylarylsiloxanes are chosen preferably from linear and/or branched polydimethylmethylphenylsiloxanes and polydimethyldiphenylsiloxanes with a viscosity of from 1×10⁻⁵ to 5×10⁻² m²/s at 25° C.

Among these polyalkylarylsiloxanes, mention may be made, by way of example, of the products sold under the following names:

the Silbione oils of the 70 641 series from Rhodia Chimie;

the oils of the Rhodorsil 70 633 and 763 series from Rhodia Chimie;

the oil Dow Corning 556 Cosmetic Grade Fluid from Dow Corning;

the silicones of the PK series from Bayer, such as the product PK20;

the silicones of the PN and PH series from Bayer, such as the products PN1000 and PH1000;

certain oils of the SF series from General Electric, such as SF 1023, SF 1154, SF 1250 and SF 1265.

The silicone gums that can be used in accordance with the invention are, preferably, polydiorganosiloxanes with high number-average molecular masses of between 200 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Mention may be made more preferably of the following products:

polydimethylsiloxane,

polydimethylsiloxane/methylvinylsiloxane gums,

polydimethylsiloxane/diphenylsiloxane,

polydimethylsiloxane/phenylmethylsiloxane,

polydimethylsiloxane/diphenylsiloxane/methylvinylsiloxane.

Products that can be used more preferably in accordance with the invention are mixtures such as:

mixtures formed from a polydimethylsiloxane hydroxylated at the chain end (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;

mixtures of two PDMSs with different viscosities, and more preferably of a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. This product preferably contains 15% SE 30 gum and 85% SF 96 oil.

The organopolysiloxane resins that can be used in accordance with the invention are crosslinked siloxane systems containing the following units: R₂SiO_{2/2}, R₃SiO_{1/2}, RSiO_{3/2} and SiO_{4/2} in which R represents a hydrocarbon-based group containing 1 to 16 carbon atoms or a phenyl group. Among these products, those particularly preferred are the ones in which R denotes a C₁-C₄ lower alkyl radical, more preferably methyl, or a phenyl radical.

Among these resins, mention may be made of the product sold under the name Dow Corning 593 or those sold under the names Silicone Fluid SS 4230 and SS 4267 by the company General Electric, which are silicones of dimethyl/trimethyl siloxane structure.

Mention may also be made of the trimethyl siloxysilicate type resins sold under the names X22-4914, X21-5034 and X21-5037 by the company Shin-Etsu.

The organomodified silicones that can be used in accordance with the invention are silicones as defined above and comprising in their structure one or more organofunctional groups attached via a hydrocarbon-based radical.

Among the organomodified silicones, mention may be made of polyorganosiloxanes comprising:

polyethyleneoxy and/or polypropyleneoxy groups optionally comprising C₆-C₂₄ alkyl groups, such as the products known as dimethicone copolyol sold by the company Dow Corning under the name DC 1248 or the oils Silwet L 722, L 7500, L 77 and L 711 by the company Union Carbide, and the (C₁₂)alkylmethicone copolyol sold by the company Dow Corning under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, preferably, C₁-C₄ aminoalkyl groups;

thiol groups such as the products sold under the names GP 72 A and GP 71 from Genesee;

alkoxylated groups such as the product sold under the name Silicone Copolymer F-755 by SWS Silicones and Abil Wax 2428, 2434 and 2440 by the company Goldschmidt;

hydroxylated groups such as the polyorganosiloxanes containing a hydroxyalkyl function, described in French patent application FR-A-85/16334,

corresponding to formula (XI):

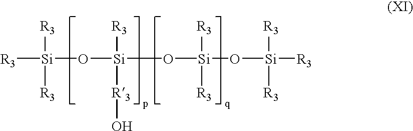

in which the radicals R₃, which may be identical or different, are chosen from methyl and phenyl radicals; at least 60 mol % of the radicals R₃ denoting methyl; the radical R₁₃ is a C₂-C₁₈ divalent hydrocarbon-based alkylene chain unit; p is between 1 and 30 inclusive; q is between 1 and 150 inclusive;

acyloxyalkyl groups such as, for example, the polyorganosiloxanes described in patent U.S. Pat. No. 4,957,732 and corresponding to formula (XII):

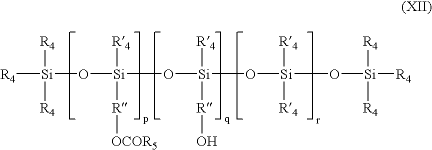

in which:

R₄ denotes a methyl, phenyl, —OCOR₅ or hydroxyl group, one of the radicals R₄ per silicon atom possibly being OH;

R′₄ denotes methyl or phenyl; at least 60 mol % of all the radicals R₄ and R₁₄ denoting methyl;

R₅ denotes C₈-C₂₀ alkyl or alkenyl;

R″ denotes a C₂-C₁₈ linear or branched divalent hydrocarbon-based alkylene radical;

r is between 1 and 120 inclusive;

p is between 1 and 30;

q is equal to 0 or is less than 0.5 p, p+q being between 1 and 30; the polyorganosiloxanes of formula (XII) may contain groups:

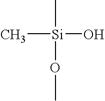

in proportions not exceeding 15% of the sum p+q+r;

anionic groups of carboxylic type, such as, for example, in the products described in patent EP 186 507 from the company Chisso Corporation, or of alkylcarboxylic type, such as those present in the product X-22-3701E from the company Shin-Etsu; 2-hydroxyalkyl sulphonate; 2-hydroxyalkyl thiosulphate such as the products sold by the company Goldschmidt under the names Abil S201 and Abil S255;

hydroxyacylamino groups, such as the polyorganosiloxanes described in patent application EP 342 834. Mention may be made, for example, of the product Q2-8413 from the company Dow Corning.

According to the invention, it is also possible to use silicones comprising a polysiloxane portion and a portion consisting of a non-silicone organic chain, one of the two portions constituting the main chain of the polymer, the other being grafted onto the said main chain. These polymers are described, for example, in patent applications EP-A-412 704, EP-A-412 707, EP-A-640 105, WO 95/00578, EP-A-582 152 and WO 93/23009 and U.S. Pat. Nos. 4,693,935; 4,728,571; and 4,972,037. These polymers are preferably anionic or nonionic.

Such polymers are, for example, copolymers that can be obtained by free-radical polymerization starting with a monomer mixture consisting of:

a) 50 to 90% by weight of tert-butyl acrylate;

b) 0 to 40% by weight of acrylic acid;

c) 5 to 40% by weight of silicone macromer of formula:

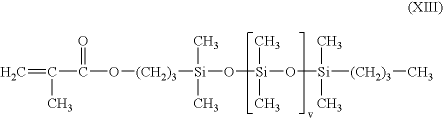

with v being a number ranging from 5 to 700; the weight percentages being calculated relative to the total weight of the monomers.

Other examples of grafted silicone polymers are, preferably, polydimethylsiloxanes (PDMS) onto which are grafted, via a connecting chain unit of thiopropylene type, mixed polymer units of poly(meth)acrylic acid type and of polyalkyl (meth)acrylate type and polydimethylsiloxanes (PDMS) onto which are grafted, via a connecting chain unit of thiopropylene type, polymer units of polyisobutyl (meth)acrylate type.

According to the invention, all of the silicones can also be used in the form of emulsions, nanoemulsions or microemulsions.

The polyorganosiloxanes that are particularly preferred in accordance with the invention are:

non-volatile silicones chosen from the family of polyalkylsiloxanes containing trimethylsilyl end groups, such as oils having a viscosity of between 0.2 and 2.5 m²/s at 25° C., such as the oils of the DC200 series from Dow Corning with a viscosity of 60 000 cSt, of the Silbione 70047 and 47 series and more preferably the oil 70 047 V 500 000, which are sold by the company Rhodia Chimie, polyalkylsiloxanes containing dimethylsilanol end groups, such as dimethiconols, or polyalkylarylsiloxanes such as the oil Silbione 70641 V 200 sold by the company Rhodia Chimie;

the organopolysiloxane resin sold under the name Dow Corning 593;

polysiloxanes containing amine groups, such as amodimethicones or trimethylsilylamodimethicones.

The cationic proteins or cationic protein hydrolysates are, preferably, chemically modified polypeptides bearing quaternary ammonium groups at the end of the chain or grafted thereto. Their molecular mass can range, for example, from 1500 to 10 000 and preferably from 2000 to 5000 approximately. Among these compounds, mention may be made preferably of:

collagen hydrolysates bearing triethylammonium groups, such as the products sold under the name Quat-Pro E by the company Maybrook and referred to in the CTFA dictionary as “Triethonium Hydrolyzed Collagen Ethosulfate”;

collagen hydrolysates bearing trimethylammonium and trimethylstearylammonium chloride groups, sold under the name Quat-Pro S by the company Maybrook and referred to in the CTFA dictionary as “Steartrimonium Hydrolyzed Collagen”;

animal protein hydrolysates bearing trimethylbenzylammonium groups such as the products sold under the name Crotein BTA by the company Croda and referred to in the CTFA dictionary as “Benzyltrimonium hydrolyzed animal protein”;

protein hydrolysates bearing, on the polypeptide chain, quaternary ammonium groups containing at least one alkyl radical having from 1 to 18 carbon atoms.

Among these protein hydrolysates, mention may be made, inter alia, of:

Croquat L in which the quaternary ammonium groups contain a C₁₂ alkyl group;

Croquat M in which the quaternary ammonium groups contain C₁₀-C₁₈ alkyl groups;

Croquat S in which the quaternary ammonium groups contain a C₁₈ alkyl group;

Crotein Q in which the quaternary ammonium groups contain at least one alkyl group having from 1 to 18 carbon atoms.

These various products are sold by the company Croda.

Other quaternized proteins or hydrolysates are, for example, those corresponding to formula (XIV):

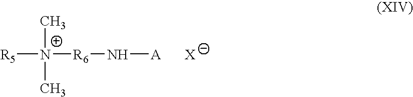

in which X⁻ is an anion of an organic or mineral acid, A denotes a protein residue derived from hydrolysates of collagen protein, R₅ denotes a lipophilic group containing up to 30 carbon atoms and R₆ represents an alkylene group having 1 to 6 carbon atoms. Mention may be made, for example, of the products sold by the company Inolex under the name Lexein QX 3000, referred to in the CTFA dictionary as “Cocotrimonium Collagen Hydrolysate”.

Mention may also be made of quaternized plant proteins such as wheat, corn or soybean proteins: as quaternized wheat proteins, mention may be made of those sold by the company Croda under the names Hydrotriticum WQ or QM, referred to in the CTFA dictionary as “Cocodimonium Hydrolysed Wheat Protein”, Hydrotriticum QL, referred to in the CTFA dictionary as “Lauridimonium Hydrolysed Wheat Protein” or Hydrotriticum QS, referred to in the CTFA dictionary as “Steardimonium Hydrolysed Wheat Protein”.

According to the present invention, the compounds of ceramide type are preferably natural or synthetic ceramides and/or glycoceramides and/or pseudoceramides and/or neoceramides.

Compounds of ceramide type are described, for example, in patent applications DE 4 424 530, DE 4 424 533, DE 4 402 929, DE 4 420 736, WO 95/23807, WO 94/07844, EP-A-0 646 572, WO 95/16665, FR-2 673 179, EP-A-0 227 994, WO 94/07844, WO 94/24097 and WO 94/10131, the teachings of which are included herein by way of reference.

Compounds of ceramide type that are particularly preferred according to the invention are, for example:

2-N-linoleoylaminooctadecane-1,3-diol,

2-N-oleoylaminooctadecane-1,3-diol,

2-N-palmitoylaminooctadecane-1,3-diol,

2-N-stearoylaminooctadecane-1,3-diol,

2-N-behenoylaminooctadecane-1,3-diol,

2-N-[2-hydroxypalmitoyl]aminooctadecane-1,3-diol,

2-N-stearoylaminooctadecane-1,3,4-triol and preferably N-stearoylphytosphingosine,

2-N-palmitoylaminohexadecane-1,3-diol,

bis(N-hydroxyethyl-N-cetyl)malonamide,

N-(2-hydroxyethyl)-N-(3-cetyloxy-2-hydroxypropyl)cetylamide,

N-docosanoyl-N-methyl-D-glucamine,

or mixtures of these compounds.

It is also possible to use cationic surfactants, among which mention may be made preferably of: optionally polyoxyalkylenated primary, secondary or tertiary fatty amine salts; quaternary ammonium salts; imidazoline derivatives; or amine oxides of cationic nature.

Examples of quaternary ammonium salts include:

those of general formula (XV) below:

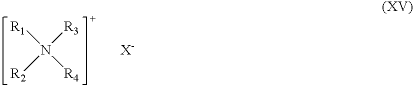

in which the radicals R₁ to R₄, which may be identical or different, represent a linear or branched aliphatic radical containing from 1 to 30 carbon atoms, or an aromatic radical such as aryl or alkylaryl. The aliphatic radicals can comprise hetero atoms such as, preferably, oxygen, nitrogen, sulphur or halogens. The aliphatic radicals are chosen, for example, from alkyl, alkoxy, polyoxy(C₂-C₆) alkylene, alkylamide, (C₁₂-C₂₂) alkylamido (C₂-C₆) alkyl, (C₁₂-C₂₂)alkylacetate and hydroxyalkyl radicals, comprising from about 1 to 30 carbon atoms; X⁻ is an anion chosen from the group of halides, phosphates, acetates, lactates, (C₂-C₆)alkyl sulphates and alkyl or alkylaryl sulphonates;

quaternary ammonium salts of imidazolinium, such as, for example, the salt of formula (XVI) below:

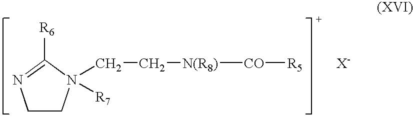

in which R₅ represents an alkenyl or alkyl radical containing from 8 to 30 carbon atoms, for example tallow fatty acid derivatives, R₆ represents a hydrogen atom, a C₁-C₄ alkyl radical or an alkenyl or alkyl radical containing from 8 to 30 carbon atoms, R₇ represents a C₁-C₄ alkyl radical, R₈ represents a hydrogen atom or a C₁-C₄ alkyl radical, X⁻ is an anion chosen from the group of halides, phosphates, acetates, lactates, alkyl sulphates and alkyl or alkylaryl sulphonates. R₅ and R₆ preferably denote a mixture of alkenyl or alkyl radicals containing from 12 to 21 carbon atoms, for example tallow fatty acid derivatives, R₇ denotes a methyl radical and R₈ denotes a hydrogen atom. Such a product is sold, for example, under the name “Rewoquat W 75” by the company Degussa;

diquaternary ammonium salts of formula (XVII):

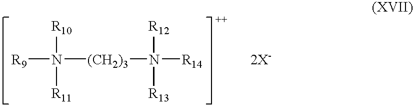

in which R₉ denotes an aliphatic radical containing from about 16 to 30 carbon atoms, R₁₀, R₁₁, R₁₂, R₁₃ and R₁₄, which may be identical or different, are chosen from hydrogen or an alkyl radical containing from 1 to 4 carbon atoms, and X⁻ is an anion chosen from the group of halides, acetates, phosphates, nitrates and methyl sulphates. Such diquaternary ammonium salts preferably comprise propane tallow diammonium dichloride;

quaternary ammonium salts containing at least one ester function.

The quaternary ammonium salts containing at least one ester function that may be used according to the invention are, for example, those of formula (XVIII) below:

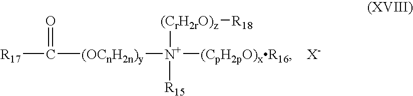

in which:

R₁₅ is chosen from C₁-C₆ alkyl radicals and C₁-C₆ hydroxyalkyl or dihydroxyalkyl radicals;

R₁₆ is chosen from:

a radical

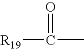

linear or branched, saturated or unsaturated C₁-C₂₂ hydrocarbon-based radicals R₂₀,

a hydrogen atom,

R₁₈ is chosen from:

a radical

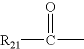

linear or branched, saturated or unsaturated C₁-C₆ hydrocarbon-based radicals R₂₂,

a hydrogen atom,

R₁₇, R₁₉ and R₂₁, which may be identical or different, are chosen from linear or branched, saturated or unsaturated C₇-C₂₁ hydrocarbon-based radicals;

n, p and r, which may be identical or different, are integers ranging from 2 to 6;

y is an integer ranging from 1 to 10;

x and z, which may be identical or different, are integers ranging from 0 to 10;

X⁻ is a simple or complex, organic or inorganic anion;

with the proviso that the sum x+y+z is from 1 to 15, that when x is 0, then R₁₆ denotes R₂₀ and that when z is 0, then R₁₈ denotes R₂₂.

The R₁₅ alkyl radicals may be linear or branched and more preferably linear.

R₁₅ preferably denotes a methyl, ethyl, hydroxyethyl or dihydroxypropyl radical and more preferably a methyl or ethyl radical.

The sum x+y+z is preferably from 1 to 10.

When R₁₆ is a hydrocarbon-based radical R₂₀, it may be long and contain from 12 to 22 carbon atoms, or short and contain from 1 to 3 carbon atoms.

When R₁₈ is a hydrocarbon-based radical R₂₂, it preferably contains 1 to 3 carbon atoms.

R₁₇, R₁₉ and R₂₁, which may be identical or different, are preferably chosen from linear or branched, saturated or unsaturated C₁₁-C₂₁ hydrocarbon-based radicals, and more preferably from linear or branched, saturated or unsaturated, C₁₁-C₂₁ alkyl and alkenyl radicals.

x and z, which may be identical or different, are preferably 0 or 1.

y is preferably equal to 1.

n, p and r, which may be identical or different, are preferably 2 or 3 and even more preferably are equal to 2.

The anion is preferably a halide (chloride, bromide or iodide) or an alkyl sulphate, more preferably methyl sulphate. However, methanesulphonate, phosphate, nitrate, tosylate, an anion derived from an organic acid, such as acetate or lactate, or any other anion that is compatible with the ammonium containing an ester function, may be used.

The anion X⁻ is even more preferably chloride or methyl sulphate.

The ammonium salts more preferably used are those of formula (XVIII) in which:

R₁₅ denotes a methyl or ethyl radical,

x and y are equal to 1;

z is equal to 0 or 1;

n, p and r are equal to 2;

R₁₆ is chosen from:

a radical

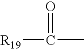

methyl, ethyl or C₁₄-C₂₂ hydrocarbon-based radicals;

a hydrogen atom;

R₁₈ is chosen from:

a radical

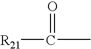

a hydrogen atom;

R₁₇, R₁₉ and R₂₁, which may be identical or different, are chosen from linear or branched, saturated or unsaturated C₁₃-C₁₇ hydrocarbon-based radicals and preferably from linear or branched, saturated or unsaturated C₁₃-C₁₇ alkyl and alkenyl radicals.

The hydrocarbon-based radicals are preferably linear.

Examples that may be mentioned include the compounds of formula (XVI) such as the diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (chloride or methyl sulphate preferably), and mixtures thereof. The acyl radicals preferably contain 14 to 18 carbon atoms and are obtained more preferably from a plant oil such as palm oil or sunflower oil. When the compound contains several acyl radicals, these radicals may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by a quaternization using an alkylating agent such as an alkyl halide (preferably a methyl or ethyl halide), a dialkyl sulphate (preferably dimethyl or diethyl sulphate), methyl methanesulphonate, methyl para-toluenesulphonate, glycol chlorohydrin or glycerol chlorohydrin.

Such compounds are sold, for example, under the names Dehyquart by the company Cognis, Stepanquat by the company Stepan, Noxamium by the company CECA or Rewoquat WE 18 by the company Degussa.

It is also possible to use the ammonium salts containing at least one ester function that are described in patents U.S. Pat. No. 4,874,554 and U.S. Pat. No. 4,137,180.

Among the quaternary ammonium salts of formula (XV), the ones that are preferred are, on the one hand, tetraalkylammonium chlorides such as, for example, dialkyldimethylammonium chlorides or alkyltrimethylammonium chlorides, in which the alkyl radical contains from about 12 to 22 carbon atoms, preferably behenyltrimethylammonium chloride, distearyldimethylammonium chloride, cetyltrimethylammonium chloride, or benzyldimethylstearylammonium chloride, or, on the other hand, stearamidopropyldimethyl(myristyl acetate)ammonium chloride sold under the name Ceraphyl 70 by the company Van Dyk.

The fatty acids are chosen more preferably from myristic acid, palmitic acid, stearic acid, behenic acid, oleic acid, linoleic acid, linolenic acid and isostearic acid.

The fatty acid derivatives are preferably carboxylic acid esters, preferably mono-, di-, tri- or tetracarboxylic esters.

The monocarboxylic acid esters are, preferably, linear or branched, saturated or unsaturated C₁-C₂₆ aliphatic acid monoesters of linear or branched, saturated or unsaturated, C₁-C₂₆ aliphatic alcohols, the total carbon number of these esters being greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isostearyl neopentanoate, isodecyl neopentanoate.

C₄-C₂₂ di- or tricarboxylic acid esters of C₁-C₂₂ alcohols and mono-, di- or tricarboxylic acid esters of C₂-C₂₆ di-, tri-, tetra- or pentahydroxy alcohols can also be used.

Mention may be made preferably of: diethyl sebacate; diisopropyl sebacate; diisopropyl adipate; di-n-propyl adipate; dioctyl adipate; diisostearyl adipate; dioctyl maleate; glyceryl undecylenate; octyldodecylstearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate dicaprate; tridecyl erucate; triisopropyl citrate; triisostearyl citrate; glyceryl trilactate; glyceryl trioctanoate; trioctyldodecyl citrate; trioleyl citrate.

Among the esters mentioned above, it is preferred to use ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate, cetyl octanoate, isostearyl neopentanoate, isodecyl neopentanoate.

The fluoro oils are, for example, the perfluoropolyethers described preferably in patent application EP-A-486 135 and the fluorohydrocarbon compounds described in patent application WO 93/11103. The teaching of these two patent applications is included in its entirety in the present application by way of reference.

The term “fluorohydrocarbon compounds” denotes compounds whose chemical structure contains a carbon skeleton in which certain hydrogen atoms have been replaced with fluorine atoms.

The fluoro oils can also be fluorocarbons such as fluoroamines, for example perfluorotributylamine, fluorohydrocarbons, for example perfluorodecahydronaphthalene, fluoro esters and fluoro ethers.

The perfluoropolyethers are sold, for example, under the trade names Fomblin by the company Montefluos and Krytox by the company Du Pont.

Among the fluorohydrocarbon compounds, mention may also be made of fluorine-containing fatty acid esters such as the product sold under the name Nofable FO by the company Nippon Oil.

Needless to say, it is possible to use mixtures of conditioning agents.

According to the invention, the conditioning agent(s) may represent from 0.001% to 20% by weight, preferably from 0.01% to 10% by weight and more preferably from 0.1% to 3% by weight, relative to the total weight of the final composition.

The compositions of the invention also preferably contain at least one surfactant, which is generally present in an amount of between 0.1% and 60% by weight approximately, preferably between 1% and 40% and even more preferably between 5% and 30%, relative to the total weight of the composition.

This surfactant may be chosen from anionic, amphoteric, nonionic surfactants, or mixtures thereof.

The surfactants that are suitable for carrying out the present invention are preferably the following:

(i) Anionic Surfactant(s):

In the context of the present invention, their nature does not represent a truly critical factor.

Thus, as examples of anionic surfactants which can be used, alone or as mixtures, in the context of the present invention, mention may be made preferably (non-limiting list) of salts (preferably alkaline salts, preferably sodium salts, ammonium salts, amine salts, amino alcohol salts or magnesium salts) of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates; alkyl sulphosuccinates, alkyl ether sulphosuccinates, alkylamide sulphosuccinates; alkyl sulphosuccinamates; alkyl sulphoacetates; alkyl ether phosphates; acyl sarcosinates; acyl isethionates and N-acyltaurates, the alkyl or acyl radical of all of these various compounds preferably containing from 8 to 24 carbon atoms, and the aryl radical preferably denoting a phenyl or benzyl group. Among the anionic surfactants which can also be used, mention may also be made of fatty acid salts such as the salts of oleic, ricinoleic, palmitic and stearic acids, coconut oil acid or hydrogenated coconut oil acid; acyl lactylates in which the acyl radical contains 8 to 20 carbon atoms. Weakly anionic surfactants can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, preferably those containing from 2 to 50 ethylene oxide groups, and mixtures thereof.

Among the anionic surfactants, it is preferred according to the invention to use alkyl sulphate salts, alkyl ether sulphate salts, or mixtures thereof.

(ii) Nonionic Surfactant(s):

The nonionic surfactants are, themselves also, compounds that are well known per se (see for example in this respect “Handbook of Surfactants” by M. R. Porter, published by Blackie & Son (Glasgow and London), 1991, pp. 116-178) and, in the context of the present invention, their nature is not a critical feature. Thus, they can be chosen preferably from (non-limiting list) polyethoxylated, polypropoxylated or polyglycerolated fatty acids, alkylphenols, α-diols or alcohols having a fatty chain containing, for example, 8 to 18 carbon atoms, it being possible for the number of ethylene oxide or propylene oxide groups to range preferably from 2 to 50 and for the number of glycerol groups to range preferably from 2 to 30. Mention may also be made of copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and preferably 1.5 to 4, glycerol groups; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides. It will be noted that the alkylpolyglycosides constitute nonionic surfactants that are particularly preferred in the context of the present invention.

(iii) Amphoteric Surfactant(s):

The amphoteric surfactants, whose nature is not a critical feature in the context of the present invention, can be, preferably (non-limiting list), aliphatic secondary or tertiary amine derivatives in which the aliphatic radical is a linear or branched chain containing 8 to 22 carbon atoms and containing at least one water-soluble anionic group (for example carboxylate, sulphonate, sulphate, phosphate or phosphonate); mention may also be made of (C₈-C₂₀) alkylbetaines, sulphobetaines, (C₈-C₂₀) alkylamido (C₁-C₆) alkylbetaines or (C₈-C₂₀) alkylamido (C₁-C₆) alkylsulphobetaines.

Among the amine derivatives, mention may be made of the products sold under the name Miranol, as described in U.S. Pat. Nos. 2,528,378 and 2,781,354 and having the structures:

R₂—CONHCH₂CH₂—N(R₃)(R₄)(CH₂COO—) (2)

in which: R₂ denotes an alkyl radical derived from an acid R₂—COOH present in hydrolysed coconut oil, a heptyl, nonyl or undecyl radical, R₃ denotes a β-hydroxyethyl group and R₄ denotes a carboxymethyl group; and

R₅—CONHCH₂CH₂—N(B)(C) (3)

in which:

B represents —CH₂CH₂OX′, C represents —(CH₂)ₙ—Y′, with z=1 or 2,

X′ denotes the —CH₂CH₂—COOH group or a hydrogen atom,

Y′ denotes —COOH or the —CH₂—CHOH—SO₃H radical,

R₅ denotes an alkyl radical of an acid RG-COOH present in coconut oil or in hydrolysed linseed oil, an alkyl radical, preferably a C₇, C₉, C₁₁ or C₁₃ alkyl radical, a C₁₇ alkyl radical and its iso form, an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of example, mention may be made of the cocoamphodiacetate sold under the trade name Miranol C2M Concentrate by the company Rhodia Chimie.

In the compositions in accordance with the invention, mixtures of surfactants are preferably used, and preferably mixtures of anionic surfactants and mixtures of anionic surfactants and of amphoteric or nonionic surfactants. One mixture that is particularly preferred is a mixture consisting of at least one anionic surfactant and of at least one amphoteric surfactant.

The anionic surfactant preferably used is chosen from sodium, triethanolamine or ammonium (C₁₂-C₁₄)alkyl sulphates, sodium, triethanolamine or ammonium (C₁₂-C₁₄)alkyl ether sulphates oxyethylenated with 2.2 mol of ethylene oxide, sodium cocoyl isethionate and sodium α-(C₁₄-C₁₆)olefin sulphonate, and mixtures thereof, with:

either an amphoteric surfactant such as the amine derivatives known as disodium cocoamphodipropionate or sodium cocoamphopropionate sold preferably by the company Rhodia Chimie under the trade name Miranol C2M CONC as an aqueous solution containing 38% active material, or under the name Miranol C32;

or an amphoteric surfactant of zwitterionic type such as alkylbetaines, preferably the cocobetaine sold under the name Dehyton AB 30 as an aqueous solution containing 32% AM by the company Cognis.

The composition of the invention may also contain at least one additive chosen from thickeners, fragrances, nacreous agents, preserving agents, anionic or nonionic polymers, non-cationic proteins, non-cationic protein hydrolysates, 18-methyleicosanoic acid, hydroxy acids, polymers other than those of the invention and preferably polyether nonionic or cationic associative polyurethanes, and any other additive conventionally used in cosmetics that does not affect the properties of the compositions according to the invention.

These additives are present in the composition according to the invention in proportions that may range from 0 to 20% by weight relative to the total weight of the composition. The precise amount of each additive is readily determined by a person skilled in the art, depending on its nature and its function.

The compositions in accordance with the invention may be used more preferably for washing or treating keratinous materials such as the hair, the skin, the eyelashes, the eyebrows, the nails, the lips or the scalp, and more preferably the hair.

Preferably, the compositions according to the invention are detergent compositions such as shampoos, shower gels and bubble baths. In this embodiment of the invention, the compositions comprise a washing base, which is generally aqueous.

The surfactant(s) forming the washing base may be chosen, without discrimination, alone or as mixtures, from the anionic, amphoteric and nonionic surfactants as defined above.

The quantity and quality of the washing base are those that are sufficient to give the final composition satisfactory foaming power and/or detergent power.

Thus, according to the invention, the washing base can represent from 4% to 50% by weight, preferably from 6% to 35% by weight and even more preferably from 8% to 25% by weight, relative to the total weight of the final composition. Preferably, the washing base contains at least 3% by weight and more preferably from 4% to 30% by weight of anionic surfactants relative to the total weight of the composition.

The pH of the composition applied to the keratinous materials is generally between 2 and 11. It is preferably between 3 and 8, and may be adjusted to the desired value by means of acidifying or basifying agents that are well known in the prior art for compositions applied to keratinous materials.

Among the basifying agents that may be mentioned, for example, are aqueous ammonia, alkali metal carbonates, alkanolamines such as monoethanolamine, diethanolamine and triethanolamine and also derivatives thereof, oxyethylenated and/or oxypropylenated hydroxyalkylamines and ethylenediamines, sodium hydroxide, potassium hydroxide and the compounds of formula (XX) below:

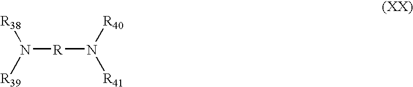

in which R is a propylene residue optionally substituted with a hydroxyl group or a C₁-C₄ alkyl radical; R₃₈, R₃₉, R₄₀ and R₄₁, which may be identical or different, represent a hydrogen atom, a C₁-C₄ alkyl radical or a C₁-C₄ hydroxyalkyl radical.

The acidifying agents are conventionally, for example, mineral or organic acids, for instance hydrochloric acid, orthophosphoric acid, carboxylic acids, for instance tartaric acid, citric acid or lactic acid, or sulphonic acids.

The physiologically and preferably cosmetically acceptable medium may consist solely of water, of a cosmetically acceptable solvent or of a mixture of water and a cosmetically acceptable solvent such as a C₁-C₄ lower alcohol, for instance ethanol, isopropanol, tert-butanol or n-butanol; polyols, for instance propylene glycol, lycerol, diethylene glycol, polyol ethers.

A subject of the invention is also a process for treating keratinous materials such as the skin or the hair, characterized in that it consists in applying to the keratinous materials a cosmetic composition as defined above, and then in optionally rinsing with water.

Thus, this process according to the invention allows holding of the hairstyle and the treatment, care or washing of or removal of makeup from the skin, the hair or any other keratin material.

The compositions of the invention may also be in the form of a rinse-out or leave-in conditioner, permanent-waving, hair-relaxing, dyeing or bleaching compositions, or alternatively in the form of rinse-out compositions to be applied before or after dyeing, bleaching, permanent-waving or relaxing the hair, or alternatively between the two steps of a permanent-waving or hair-relaxing operation.

The compositions of the invention may also be in the form of washing compositions for the skin, preferably in the form of bath or shower solutions or gels or makeup-removing products.

The compositions according to the invention may also be in the form of aqueous or aqueous-alcoholic lotions for skincare and/or haircare.

The cosmetic compositions according to the invention may be in the form of a gel, a milk, a cream, an emulsion, a thickened lotion or a mousse and may be used for the skin, the nails, the eyelashes, the lips and, more preferably, the hair.

The compositions may be packaged in various forms, preferably in vaporizers, pump-dispenser bottles or in aerosol containers to allow the composition to be applied in vaporized form or in the form of a mousse. Such packaging forms are indicated, for example, when it is desired to obtain a spray, a lacquer or a mousse for treating the hair.

Throughout the text hereinabove and hereinbelow, the percentages expressed are on a weight basis.

The invention will now be illustrated more fully with the aid of the examples that follow, which cannot be considered as limiting it to the embodiments described.

In the examples, AM means active material.

### EXAMPLE 1

A shampoo composition is prepared:

| | in g |
|---|---|
| Guar hydroxypropyltrimonium chloride | 0.05 |
| Cocobetaine (32% AM) | 9 |
| Sodium lauryl ether (2 EO) sulphate (70% AM) | 22.2 |
| Sodium methyl paraben | 0.2 |
| DMDM hydantoin | 0.25 |
| Mucic acid (galactaric acid) | 0.3 |
| Dimethicone (DC 200 fluid 300000 | 2.7 |
| from Dow Corning) | |
| Mixture of cetyl alcohol and of 1-(hexadecyloxy)- | 2.5 |
| 2-octadecanol | |
| Fragrance | 0.5 |
| Coconut monoisopropanolamide (cocamide MIPA) | 0.3 |
| Carbomer | 0.2 |
| Sodium hydroxide qs | pH 7 |
| Water qs | 100 |

Hair treated with this shampoo is supple and manageable.

### EXAMPLE 2

Ready-to-use oxidizing compositions based on aqueous hydrogen peroxide solution, for use in the dyeing, bleaching and permanent reshaping of keratin fibers.

Two ready-to-use oxidizing compositions—B and C, respectively—for the dyeing, bleaching and permanent reshaping of keratin fibers are prepared. Their qualitative and quantitative composition is given in Table I below, in which the amounts of the various constituents are expressed in grams.

TABLE I

| | Composition B | Composition C |
|---|---|---|
| Constituents | (g) | (g) |
| Cetyl alcohol | 3 | 3 |
| Sodium lauryl sulphate | 0.5 | 0.5 |
| Polyglycolated oleyl | 0.45 | 0.45 |
| alcohol (2 mol) | | |
| Polyglycerolated oleyl | 0.35 | 0.35 |
| alcohol (4 mol) | | |
| Simethicone | 0.045 | 0.045 |
| Mucic acid* | 0.02 | 0.03 |
| Tetrasodium pyrophosphate | 0.02 | 0.04 |
| 10H₂O | | |
| Sodium salicylate | — | 0.035 |
| Sodium stannate | 0.04 | — |
| 50% aqueous hydrogen | 24 | 18 |
| peroxide solution | | |
| Aqueous 85% phosphoric | qs pH = 2 | qs pH = 2 |
| acid solution | | |
| Water | qs 100 g | qs 100 g |

*Muciliance - from the company Soliance

### EXAMPLE 3

Two compositions—D and E, respectively—for use in bleaching are prepared, these compositions both being anhydrous compositions in pulverulent form, comprising a complexing agent in accordance with the invention.

Table II shows the qualitative and quantitative compositions of these compositions, the amounts being expressed in percentages by mass.

TABLE II

| | Composition | Composition E |
|---|---|---|
| Constituents | D (%) | (%) |
| Potassium persulphate | 39.5 | 46 |
| Sodium persulphate | 30 | 15 |
| Sodium disilicate | — | 15 |
| Sodium metasilicate | 14 | 4 |
| Ammonium chloride | 6 | 4 |
| Urea | — | 4.5 |
| Mucic acid* | 1 | 0.8 |
| Hexamethyl diisocyanate/- | 0.5 | — |
| polyethylene glycol | | |
| copolymer containing α and | | |
| ω stearyl polyoxyethylene | | |
| end groups** | | |
| Acrylic acid/alkyl | — | 1 |
| methacrylate (C10/C30) | | |
| crosslinked copolymer*** | | |
| Weakly crosslinked | — | 2 |
| carboxymethyl potato | | |
| starch/sodium salt | | |
| Guar gum | 2 | 1.5 |
| Dye (ultramarine) | 0.5 | — |
| Titanium oxide | 0.5 | 1 |
| Sodium lauryl sulphate | 2 | 2 |
| Calcium stearate | 1 | 1 |
| Fumed silica of | 3 | 0.2 |
| hydrophilic nature | | |
| Hydrogenated | — | 2 |
| polydecene**** | | |

*Muciliance - from the company Soliance
**SER-AD FX 1100 - from the company Servo Delden
***Carbopol ETD 2020 - from the company Noveon
****Silkflo 366 NF Polydecene - from the company Amoco

Chemical

The bleaching composition D (40 g) is mixed with the oxidizing composition based on aqueous hydrogen peroxide solution, A (80 g). The ready-to-use bleaching mixture thus obtained is applied for 45 minutes, under a hood, to natural dark hair, and is then rinsed out thoroughly with water. After these operations, a strong, uniform bleaching result is obtained.

The bleaching composition E (40 g) is mixed with the oxidizing composition based on aqueous hydrogen peroxide solution, C (60 g). The ready-to-use bleaching mixture thus obtained is applied for 30 minutes, under a hood, to natural dark hair and is then rinsed out thoroughly with water. After these operations, a strong, uniform bleaching result, with soft, shiny hair that is easy to disentangle, is obtained.

### EXAMPLE 4

Two compositions —F and G, respectively—intended for use in bleaching were prepared in this example, these compositions both being anhydrous compositions in paste form comprising a complexing agent in accordance with the invention.

Table III shows the qualitative and quantitative compositions of these compositions, the amounts being expressed in percentages by mass.

TABLE III

| | Composition | Composition |
|---|---|---|
| Constituents | F (%) | G (%) |
| Potassium persulphate | 35.8 | 35.6 |
| Sodium persulphate | 6 | 6 |
| Sodium disilicate | 15 | 15 |
| Sodium metasilicate | 3 | 3 |
| Ammonium chloride | 4.2 | 4.2 |
| Mucic acid* | 1 | 1 |
| Hexamethyl diisocyanate/- | 2 | 0.5 |
| polyethylene glycol | | |
| copolymer containing α and ω | | |
| stearyl polyoxyethylene end | | |
| groups** | | |
| Acrylic acid/alkyl | — | 0.5 |
| methacrylate (C10/C30) | | |
| crosslinked copolymer*** | | |
| Weakly crosslinked | 2 | 1 |
| carboxymethyl potato starch/ | | |
| sodium salt | | |
| Guar gum | — | 2 |
| Dye (ultramarine) | 0.5 | 0.5 |
| Titanium oxide | 1 | 1 |
| Sodium lauryl sulphate | 3.5 | 3.5 |
| Calcium stearate | 2 | 2 |
| Fumed silica of hydrophilic | 0.5 | 0.5 |
| nature | | |
| Isopropyl palmitate | 22.5 | — |
| Beeswax | 1 | — |
| Hydrogenated polydecene**** | — | 23 |
| Fumed silica of hydrophobic | — | 0.7 |
| nature | | |

*Muciliance - from the company Soliance
**SER-AD FX 1100 - from the company Servo Delden
***Carbopol ETD 2020 - from the company Noveon
****Silkflo 366 NF Polydecene - from the company Amoco

Chemical

The bleaching composition F (40 g) is mixed with the oxidizing composition based on aqueous hydrogen peroxide solution, A (80 g). The ready-to-use bleaching mixture thus obtained is applied for 45 minutes, under a hood, to natural dark hair and is then rinsed out thoroughly with water. After these operations, a strong, uniform bleaching result, with soft, shiny hair that is easy to disentangle, is obtained.

The bleaching composition G (40 g) is mixed with the oxidizing composition based on aqueous hydrogen peroxide solution, C (60 g). The ready-to-use bleaching mixture thus obtained is applied for 25 minutes, under a hood, to natural dark hair and is then rinsed out thoroughly with water. After these operations, a strong, uniform bleaching result, with soft, shiny hair that is easy to disentangle, is obtained.

### EXAMPLE 5

In this example, a reducing composition is prepared in a first stage.

Table IV shows the qualitative and quantitative compositions of these compositions, the amounts being expressed in percentages by mass.

TABLE IV

| | Composition H |
|---|---|
| Constituents | (% or g?) |
| Thioglycolic acid | 9.2 |
| Arginine | 15 |
| 20% aqueous ammonia | 1.86 |
| Ammonium carbonate | 4.5 |
| Cocoylamidopropylbetaine/glyceryl | 1.3 |
| monolaurate (25/5) as an aqueous 30% | |
| solution | |
| Peptizer | 0.8 |
| Isostearyl alcohol | 12 |
| Complexing agent | 0.4 |
| Fragrance | 0.4 |
| Water | qs 100 g |

Composition H is applied to a lock of wet hair wound beforehand on a roller 9 mm in diameter, the leave-in time being 10 minutes. The locks thus treated are then rinsed thoroughly with water.

In a second stage, an oxidizing composition, referred to as composition I, is prepared.

Table V shows the qualitative and quantitative compositions of these compositions, the amounts being expressed in percentages by mass.

TABLE V

| | Composition I |
|---|---|
| Constituents | (g) |
| Cetyl alcohol | 3 |
| Sodium lauryl sulphate | 0.5 |
| Polyglycerolated oleyl alcohol (2 mol) | 0.45 |
| Polyglycerolated oleyl alcohol (4 mol) | 0.35 |
| Simethicone | 0.045 |
| Mucic acid* | 0.03 |
| Tetrasodium pyrophosphate decahydrate | 0.02 |
| Sodium salicylate | — |
| Sodium stannate hexahydrate | 0.04 |
| 50% aqueous hydrogen peroxide solution | 5.4 |
| Aqueous 85% phosphoric acid solution | qs pH = 2 |
| Water | qs 100 g |

*Muciliance - from the company Soliance

Composition I is applied to the locks previously treated with the reducing composition H, by applying the said composition S for a leave-in time of 10 minutes. The locks are then rinsed thoroughly with water. Finally, the hair is unwound from the roller and then dried. The lock thus treated is wavy and soft.

### EXAMPLE 6

In this example, a dye composition J is prepared in a first stage.

Table VI shows the qualitative and quantitative compositions of this composition, the amounts being expressed in percentages by mass.

TABLE VI

| | Composition J |
|---|---|
| Constituents | (%) |
| para-Phenylenediamine | 0.108 |
| 2-Methyl-5-aminophenol | 0.123 |
| Ethanol | 20 |
| Benzyl alcohol | 2 |
| (C8/C10)alkyl polyglucoside as an | 3.6 |
| aqueous 60% solution* | |
| Polyethylene glycol containing 8 mol of | 3 |
| ethylene oxide | |
| Sodium metabisulphite as an aqueous 35% | 3 |
| solution | |
| Complexing agent | 0.227 |
| 20% aqueous ammonia | 0.48 |
| Water | 6.8 |

The oxidizing composition K is prepared in a second stage.

Table VII shows the qualitative and quantitative compositions of this composition, the amounts being expressed in percentages by mass.

TABLE VII

| | Composition A |
|---|---|
| Constituents | (%)? |
| Cetyl alcohol | 3 |
| Sodium lauryl sulphate | 0.5 |
| Polyglycerolated oleyl alcohol (2 mol) | 0.45 |
| Polyglycerolated oleyl alcohol (4 mol) | 0.35 |
| Simethicone | 0.045 |
| Mucic acid* | 0.1 |
| Tetrasodium pyrophosphate decahydrate | 0.02 |
| Sodium salicylate | — |
| Sodium stannate | 0.04 |
| 50% aqueous hydrogen peroxide solution | 24 |
| Aqueous 85% phosphoric acid solution | Qs pH = 2 |
| Water | Qs 100 g |

*Muciliance - from the company Soliance
**Gluconal ® GA-50-SG - from the company Akzo Nobel

The dye composition J is mixed with the oxidizing composition based on aqueous hydrogen peroxide solution, K, in equal weight.

The resulting composition is applied for 30 minutes to locks of natural grey hair containing 90% white hairs, and the hair thus treated is then rinsed thoroughly, washed with a standard shampoo, rinsed again and then dried.

### EXAMPLE 7

The following formulation, in cream form, is prepared:

| | |
|---|---|
| Cetylstearyl alcohol | 13% |
| Oxyethylenated (12 EO) lauryl alcohol | 8% |
| Oxyethylenated (3 EO) decyl alcohol at 90% | 6% |
| in water [Empilan KA 2.5/90 FL - | |
| sold by Hunstmann] | |
| Oxyethylenated (30 EO) oleocetyl alcohol | 4% |
| Lauric acid | 5% |
| Monoethanolamine | 2% |
| Propylene glycol | 8% |
| Tetramethylhexamethylenediamine/1,3- | 1% |
| dichloropropylene polycondensate at 60% in | |
| water [Mexomere PO - sold by Chimex] | |
| Dimethyldiallylammonium chloride/acrylic | 2% |
| acid copolymer (80/20) at 40.5% in water | |
| [Merquat 280 - sold by Nalco] | |
| Glycol distearate | 4% |
| Fumed silica of hydrophobic nature | 2% |
| Crosslinked polyacrylic acid [Carbopol 980 - | 0.6% |
| sold by Noveon] | |
| Mucic acid | 1% |
| 1,3-Dihydroxybenzene (resorcinol) | 0.67% |
| para-Phenylenediamine | 0.88% |
| 5-N-(β-Hydroxyethyl)amino-2-methylphenol | 0.055% |
| 2-Methyl-1,3-dihydroxybenzene | 0.11% |
| para-Aminophenol | 0.27% |
| 4-(Methylamino)-phenol hemisulphate | 0.26% |
| 1-Hydroxy-3-aminobenzene | 0.16% |
| Antioxidant | qs |
| Reducing agent | qs |
| Fragrance | qs |
| Aqueous ammonia (20% NH₃) | 11.1% |
| Water | qs |
| | 100% |

The percentages are expressed on a weight basis.

At the time of use, this composition is mixed with one and a half times its weight of an oxidizing composition with a hydrogen peroxide titre of 6%, and the mixture obtained is applied to the hair for 30 minutes and then rinsed out.

A relatively unselective shade is obtained.

1. A composition for treating a keratinous material comprising:
(a) a cosmetically acceptable medium,
(b) at least one hydroxycarboxylic acid or a salt thereof of formula (I):
R—(CHOH)₄—CO₂X (I),
wherein,
R represents a CO₂X group, and
X represents a hydrogen atom or a monovalent or divalent cation derived from a transition metal, alkali metal, alkaline-earth metal, organic amine or ammonium ion, and,
(c) at least one of protective agent and conditioning agent,
wherein said conditioning agent is a synthetic oil, plant oil, fluoro or perfluoro oil, natural or synthetic wax, silicone, non-polysaccharide cationic polymer, compound of ceramide type, cationic surfactant, fatty amine, or fatty acid or a derivative or mixture thereof.

2. The composition of claim 1, wherein said keratinous material is hair.

3. The composition of claim 1, wherein said monovalent or divalent cation is an alkali metal cation, alkaline-earth metal cation or divalent transition metal cation.

4. The composition of claim 1, wherein said at least one hydroxycarboxylic acid or a salt thereof of formula (I) is mucic acid, glucaric acid, mannaric acid, an alkali metal salt thereof, an alkaline-earth metal salt thereof, a transition metal salt thereof, or a mixture thereof.

5. The composition of claim 4, wherein said at least one hydroxycarboxylic acid or a salt thereof of formula (I) is mucic acid.

6. The composition of claim 1, wherein the content of said at least one hydroxycarboxylic acid or a salt thereof of formula (I) is from 0.001% to 10% by weight relative to the total weight of the composition.

7. The composition of claim 6, wherein said content of said at least one hydroxycarboxylic acid or a salt thereof of formula (I) is from 0.001% to 5% by weight relative to the total weight of the composition.

8. The composition of claim 1, wherein said protective agent is an organic UV-screening agent, free-radical scavenger, antioxidant, vitamin or provitamin.

9. The composition of claim 8, wherein said organic UV-screening agent is a silicone or non-silicone, water-soluble, liposoluble or water-insoluble organic screening agent or mineral oxide nanoparticles wherein the surface of said mineral oxide nanoparticles has been optionally treated.

10. The composition of claim 9, wherein said surface of said mineral oxide nanoparticles has been treated to be hydrophilic or hydrophobic.

11. The composition of claim 9, wherein said water-soluble organic UV-screening agent is para-aminobenzoic acid or its salt, anthranilic acid or its salt, salicylic acid or its salt, p-hydroxycinnamic acid or its salt, sulphonic derivative of benzoxazoles or salt thereof, sulphonic derivative of benzophenone or salt thereof, sulphonic derivative of benzylidenecamphor or salt thereof, benzylidenecamphor derivative substituted with a quaternary amine or salt thereof, phthalylidene-camphorsulphonic acid derivative or salts thereof, sulphonic derivative of benzotriazole, hydrophilic polymer with a UV-photoprotective property, or a mixture thereof.

12. The composition of claim 9, wherein said liposoluble organic UV-screening agent is a p-aminobenzoic acid derivative, salicylic acid derivative, dibenzoylmethane derivative, diphenylacrylate derivative, benzofuran derivative, polymeric UV-screening agent containing one or more organosilicon residue, cinnamic acid ester, camphor derivative, trianilino-s-triazine derivative, urocanic acid ethyl ester, benzotriazole, hydroxyphenyltriazine derivative, bis-resorcinol-dialkylaminotriazine, or a mixture thereof.

13. The composition of claim 12, wherein said p-aminobenzoic acid derivative is a p-aminobenzoic acid ester or amide.

14. The composition of claim 12, wherein said salicylic acid derivative is a salicylic acid ester.

15. The composition of claim 12, wherein said liposoluble organic UV-screening agent is octyl salicylate, 2-hydroxy-4-methoxybenzophenone, 4-tert-butyl-4′-methoxydibenzoylmethane, octocrylene, 2-ethylhexyl 4-methoxycinnamate, or the compound of formula (II)

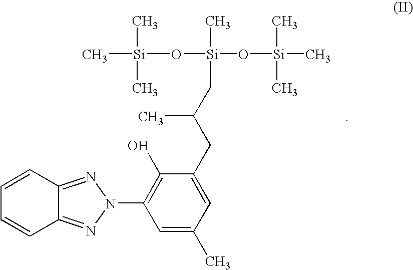

16. The composition of claim 1, wherein said synthetic oil is polyolefin of hydrogenated or non-hydrogenated polybutene type or of hydrogenated or non-hydrogenated polydecene type.

17. The composition of claim 1, wherein said cationic polymer is a polymer comprising at least one primary, secondary, tertiary or quaternary amine group that is either a part of the main polymer chain or a side substituent directly attached thereto.

18. The composition of laim 17, wherein said cationic polymer is a cationic cyclopolymer, quaternary vinylpyrrolidone polymer, quaternary vinylimidazole polymer, or a mixture thereof.

19. The composition of claim 18, wherein said cyclopolymer is a homopolymer of diallyldimethylammonium chloride, or a copolymer of diallyldimethylammonium chloride or of acrylamide.

20. The composition of claim 1, wherein said silicone is polyorganosiloxane wherein said polyorganosiloxane is insoluble in said composition.

21. The composition of claim 20, wherein said silicone is non-volatile polyorganosiloxane chosen from polyalkylsiloxane, polyarylsiloxane, polyalkylarylsiloxane, silicone gum, silicone resin, polyorganosiloxane modified with an organofunctional group, or a mixture thereof.

22. The composition of claim 21, wherein
(a) said polyalkylsiloxane is:
polydimethylsiloxane containing a trimethylsilyl end group
polydimethylsiloxane containing a dimethylsilanol end group or
polyalkyl (C₁-C₂₀) siloxane;
(b) said polyalkylarylsiloxane is:
linear and/or branched polydimethylmethylphenylsiloxane or polydimethyldiphenylsiloxane with a viscosity of between 1×10⁻⁵ and 5×10⁻² m²/s at 25° C.;
(c) said silicone gum is polydiorganosiloxane with average molecular weight of between 200 000 and 1 000 000, wherein said polydiorganosiloxane is used alone or in the form of a mixture in a solvent;
(d) said silicone resin is the group consisting of R₃SiO_{1/2}, R₂SiO_{2/2}, RSiO_{3/2} and SiO_{4/2}, wherein R represents a hydrocarbon-based group containing 1 to 16 carbon atoms or a phenyl group; and
(e) said polyorganosiloxane modified with an organofunctional group is polyorganosiluxane comprising at least one organofunctional group attached via a hydrocarbon-based radical.

23. The composition of claim 22, wherein said silicone gum is:
polydimethylsiloxane,
polydimethylsiloxane/methylvinylsiloxane,
polydimethylsiloxane/diphenylsiloxane,
polydimethylsiloxane/phenylmethylsiloxane,
polydimethylsiloxane/diphenylsiloxane/methylvinylsiloxane,
a mixture of polydimethylsiloxane hydroxylated at the chain end and cyclic polydimethylsiloxane,
a mixture of polydimethylsiloxane gum and cyclic silicone or
a mixture of polydimethylsiloxanes with different viscosities.

24. The composition of claim 22, wherein said polyorgansilxane modified within an organofunctional group is polyorganosiloxane comprising:
a) a polyethyleneoxy group;
b) a polypropyleneoxy group;
c) a substituted amine group;
d) an unsubstituted amine group;
e) a thiol group;
f) an alkoxylated group;
g) a hydroxyalkyl group;
h) an acyloxyalkyl group;
i) an alkylcarboxylic group;
j) a 2-hydroxyalkylsulphonate group;
k) a 2-hydroxyalkylthiosulphonate group; or
l) a hydroxyacylamino group.

25. The composition of claim 22, wherein said polyalkylsiloxane is polyorganosiloxane comprising:
a) a trimethylsilyl end group,
b) a dimethylsilanol end group,
c) polyalkylarylsiloxane
d) a mixture of two PDMSs consisting of a gum and an oil with different viscosities,
e) a mixture of organosiloxanes or
f) a mixture of cyclic silicone and organopolysiloxane resin.

26. The composition of claim 1, wherein said compound of ceramide type is
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol and in particular N-stearoylphytosphingosine,
2-N-palmitoylaminohexadecane-1,3-diol,
bis(N-hydroxyethyl-N-cetyl)malonamide,
N-(2-hydroxyethyl)-N-(3-cetyloxy-2-hydroxypropyl)cetylamide,
N-docosanoyl-N-methyl-D-glucamine, or a mixture thereof.

27. The composition of the claim 1, wherein said protective or conditioning agent is present in a concentration from 0.001% to 20% relative to the total weight of the composition.

28. The composition of claim 27, wherein said concentration is from 0.01% to 10% by weight relative to the total weight of the composition.

29. The composition of claim 1, further comprising at least one surfactant chosen from anionic, nonionic, amphoteric surfactant or a mixture thereof.

30. The composition of claim 29, wherein said at least one surfactant is present in a concentration from 0.1% to 60% by weight relative to the total weight of the composition.

31. The composition of claim 30, wherein said concentration is from 1% to 40% by weight relative to the total weight of the composition.

32. The composition of claim 31, wherein said concentration is from 5% to 30% by weight relative to the total weight of the composition.

33. The composition of claim 1, wherein said composition is a shampoo, a conditioner, a composition for permanent-waving, relaxing, dyeing or bleaching the hair, a rinse-out composition to be applied before and/or after a dyeing or bleaching operation, a rinse-out composition to be applied before and/or between the two steps of a permanent-waving or hair-relaxing operation, or a washing composition for the body.

34. A method of washing a keratinous material comprising applying said composition of claim 1 to said keratinous material and then rinsing said composition from said keratinous material with water.

35. A method of treating a keratinous material comprising applying said composition of claim 1 to said keratinous material and then optionally rinsing with water.
